(19) United States
(12) Patent Application Publication
(10) Pub. No.: US 2004/0120989 A1
(43) Pub. Date: Jun. 24, 2004

(54) Article for debridement and detoxification of wounds

(75) Inventors: Graydon Vadas, Charleston, SC (US); Charley Hendrix, Savannah, GA (US); Michael A. Warshaw, Savannah, GA (US)

Correspondence Address: AKERMAN SENTERFITT, P.O. BOX 3188, WEST PALM BEACH, FL, 33402-3188, US

(73) Assignee: Remedy Marketing, Inc., Charleston, SC

(21) Appl. No.: 10/620,521

(22) Filed: Jul. 16, 2003

Related U.S. Application Data
(63) Continuation-in-part of application No. 10/342,082, filed on Jan. 14, 2003.
Application No. 10/342,082 is a continuation-in-part of application No. 09/940,007, filed on Aug. 27, 2001, now abandoned.
Application No. 09/940,007 is a continuation of application No. 09/222,111, filed on Dec. 29, 1998, now abandoned.

(51) Int. Cl. A61K 9/70

(52) U.S. Cl. 424/443; 424/94.63; 424/744

(57) ABSTRACT

An abrasive pad for delivering a skin treatment for the treatment of animal and marine stings. The pad includes a material having an abrasive surface, the surface being sufficiently abrasive to mechanically abrade at least the stratum corneum from a patient's skin. A treatment solution is applied to the pad for contact with the patient's skin. The treatment solution includes 65.0% to 98.0% aloe vera and 0.1% to 20.0% of at least one proteolytic enzyme. The treatment solution contains substantially no added water and the at least one proteolytic enzyme can include papain.

### CROSS-REFERENCE TO RELATED APPLICATIONS

This application is a continuation-in-part of U.S. patent application Ser. No. 10/342,082, filed Jan. 14, 2003, which is a continuation-in-part of U.S. patent application Ser. No. 09/940,007, filed Aug. 27, 2001, which is a continuation of U.S. patent application Ser. No. 09/222,111, filed Dec. 29, 1998.

### TECHNICAL FIELD

This invention relates to an article for the treatment of bites, stings or wounds caused by animals and insects such as fire ants, jellyfish, sea lice, related arthropods and cnidarians, as well as other biologically-caused wounds and envenomations.

### BACKGROUND OF THE INVENTION

Fire ants (Solenopsis Invicta), sea lice (one of 1000 species of cnidarians), and related arthropods and cnidarians and other species are prevalent in tropical and sub-tropical areas, and can cause painful bites and stings. Fire ants in particular behave very aggressively, and can inflict very painful and toxic stings. The fire ant stings have been known to be fatal to humans, particularly as S. Invicta releases an airborne chemical signal which triggers all nearby fire ants to begin to attack the same victim, causing multiple stings. The sting, which is extremely painful to the recipient, can form a raised pustule which can rupture and become infected, leading to scarring.

Sea lice, which are the larval stage of the thimble jellyfish (Linuche Unguiculata) and stings from other cnidarians jellyfish) can also cause painful stings, which if left untreated can cause a rash and blistering of the affected area, due to nematocysts left in the wound which continue to release toxins.

Current treatments for envenomation by fire ants and cnidarians are similar, and are typically topical measures such as the application of a cortisone cream, colloidal preparation or calamine solution. Vinegar, alcohol and meat tenderizer are also recommended. In more severe cases, antihistamines, steroids, and epinephrine may be administered by I.M., I.V., and/or orally.

There are a few instances noted in the literature of remedies for stings, particularly for jellyfish stings, and several noted in“Dangerous Marine Animals” by Dr. Bruce W. Halsted of the World Life Research Institute are of interest. Halstead on several occasions refers to using sand or adhesive tape to strip the wound site after a sting. There are also recommendations to use a razor with shaving cream to strip the outer layers of the wound for treatment. Alternative treatment recommendations include forming a mud or sand paste, and using a sharp edge such as the edge of a shell or a piece of wood to scrape the wound and thus remove nematocysts. Other recommendations include using tweezers, a towel, rag, seaweed or a stick to carefully remove nematocysts from wounds.

Physicians have long used dermabrasion to treat problems ranging from acne to burns, in order to speed up cell renewal and turnover. However, delivery systems for medicaments tend to involve deep delivery of medicaments, such as by hypodermic needle. For the treatment of toxins delivered by Solenopsis Invicta and other stinging animals, a shallow delivery system is needed.

### SUMMARY OF THE INVENTION

One aspect of the invention relates to an abrasive pad for delivering a skin treatment for the treatment of animal and marine stings. The pad includes a material having an abrasive surface, where the surface is sufficiently abrasive to mechanically abrade at least the stratum corneum from a patient's skin. A treatment solution is applied to the material for contact with the patient's skin. The treatment solution includes a solution of 65.0% to 98.0% aloe vera and 0.1% to 20.0% of at least one proteolytic enzyme. The treatment solution contains substantially no added water.

The pad may be multilayered. In one arrangement, the pad may include an absorbent material carrying the treatment solution, and a porous abrasive material. In another arrangement, the pad may include a burstable receptacle containing the treatment. solution. The pad may include three layers including two outer layers and a central layer, where at least one of the outer layers can be an abrasive material and the burstable receptacle can form the central layer. A plurality of burstable receptacles may be provided, where at least one of the burstable receptacles can contain a different treatment solution from at least one other of the burstable receptacles.

The abrasive surface can be formed of matted non-woven fibers. Alternatively, the abrasive surface can be formed of a loop fabric and/or may be sponge-like. In another arrangement, the pad can be a mitt adapted to be worn over a hand of a user.

In one embodiment, the proteolytic enzyme can form 0.1% to 5.0% of the treatment solution. Preferably, the treatment solution contains 0.9% to 1.1% of the at least one proteolytic enzyme. The at least one proteolytic enzyme can be papain. In another embodiment, the aloe vera can form 90.0% to 98% of the solution. At least one humectant, such as glycerin, can form 1.0% to 10.0% of the solution. In still another embodiment, 0.1% to 5.0% of the solution can be at least one viscosity agent, such as sodium chloride and xanthan gum. At least one pH stabilizer, such as aminomethylpropanol, can form 0.05% to 1.0% of the solution and 0.05% to 1.1% of the-solution can be at least one anti-bacterial agent, such as diazolidinyl urea and triclosan. In another arrangement, 0.05% to 0.25% of the solution can be at least one fungal agent, such as methylparaben and prbpylparaben. The solution can include at least one surfactant, at least one antitoxin, at least one antiseptic, and at least one alcohol.

In one arrangement, the sting may have been caused by an arthropod. The arthropod can be Solenopsis Invicta. Alternatively, the sting may have been caused by a cnidarian. The cnidarian may be a larval of the thimble jellyfish.

Another aspect of the invention relates to a solution for treating animal and marine stings wherein the treatment solution includes a solution of 65.0% to 98.0% aloe vera and 0.1% to 20.0% of at least one proteolytic enzyme. The treatment solution contains substantially no added water. In one embodiment, the at least one proteolytic enzyme can form 0.1% to 5.0% of the treatment solution. Preferably, 0.9% to 1.1% of the solution can be the at least one proteolytic enzyme. The at least one proteolytic enzyme can be papain. In another embodiment, the aloe vera can form 90.0% to 98% of the solution. At least one humectant, such as glycerin, can form 1.0% to 10.0% of the solution. In another embodiment, 0.1% to 5.0% of the solution can be at least one viscosity agent, such as sodium chloride and xanthan gum. At least one pH stabilizer, such as aminomethylpropanol, can form 0.05% to 1.0% of the solution and 0.05% to 1.1% of the solution can be at least one anti-bacterial agent, such as diazolidinyl urea and triclosan. In another arrangement, 0.05% to 0.25% of the solution can be at least one fungal agent, such as methylparaben and propylparaben. The solution can include at least one surfactant, at least one antitoxin, at least one antiseptic, and at least one alcohol.

### BRIEF DESCRIPTION OF THE DRAWINGS

There are shown in the drawings embodiments which are presently preferred, it being understood, however, that the invention is not limited to the precise arrangements and instrumentalities shown.

FIG. 1 illustrates the length of a typical Solenopsis Invicta stinger.

FIG. 2 illustrates an armed nematocyst.

FIG. 3 illustrates a discharged nematocyst.

FIG. 4 illustrates the average human epidermal layer of skin.

FIG. 5 is a side perspective view of a pad containing a solution according to the present invention.

### DETAILED DESCRIPTION

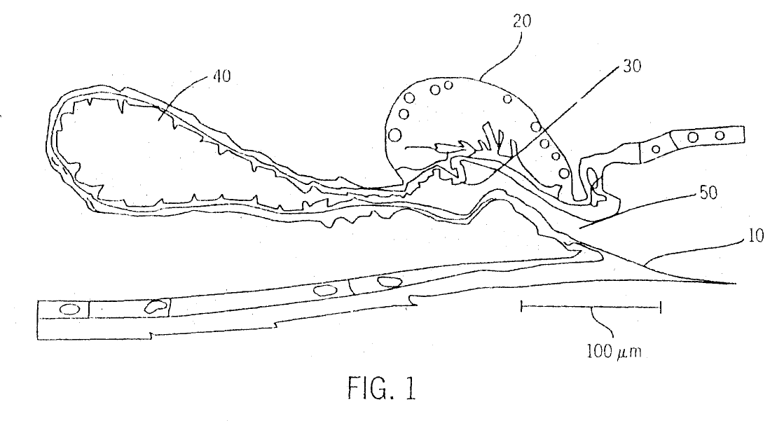

Solenopsis Invicta is a specific group of ants that are known for their aggressive behavior and their extremely painful and dangerous stings, which have been known to be fatal. A characteristic of Solenopsis Invicta and a number of other stinging arthropods is that their toxins, although powerful, are largely deposited in shallow wound sites, due to the small size of the stinger. As illustrated in FIG. 1, the stinger typically has a length of about 100 microns. The glandular epithelium 20 deposits or emits toxin 30 to a reservoir 40. The stinger 10 opens a hole or wound opening in the top layer of skin, the epidermis, of a recipient of about one-half to two-thirds of the length of the stinger and the toxin 30 enters the wound opening or hole via passage 50.

Besides arthropods, the invention is applicable for cnidarians and other biological envenomations (for example Coelenterates) stings. The cnidarians include hydras, men o'war, jellyfish, sea anemones, hydrodroids, corals, bryozoans and the like. The different varieties of cnidarians typically have similar stinging cells, referred to as nematocysts, which contain a nematocyte and are usually present on long tentacles. The nematocyte consists of a coiled tube that can be bathed in venom. When a nematocyst is triggered by some object the tube rapidly fires, embedding itself into the target, typically for food acquisition and/or for defensive purposes. Venom on the outside of the tube is deposited on the tissue of the target and can possibly enter the cardiac system of the target.

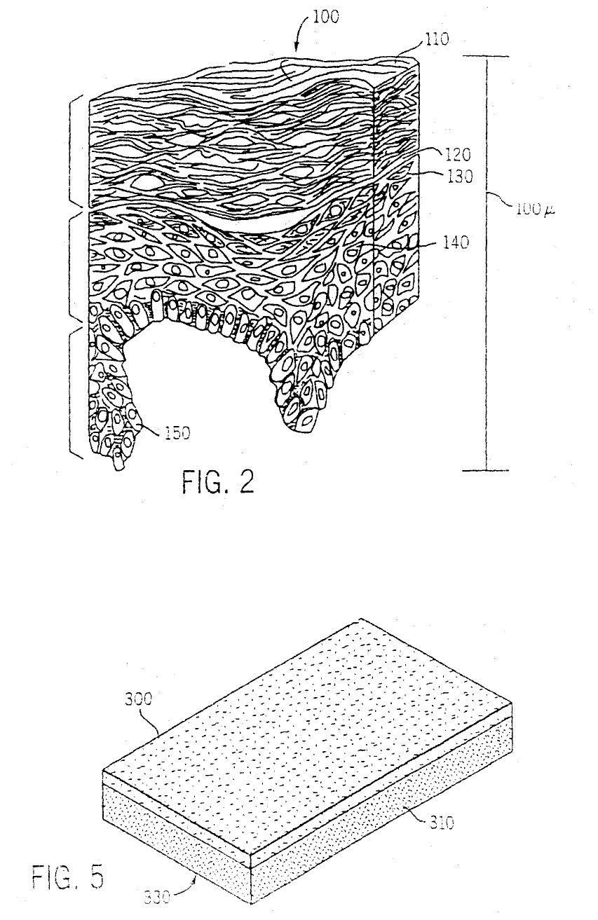

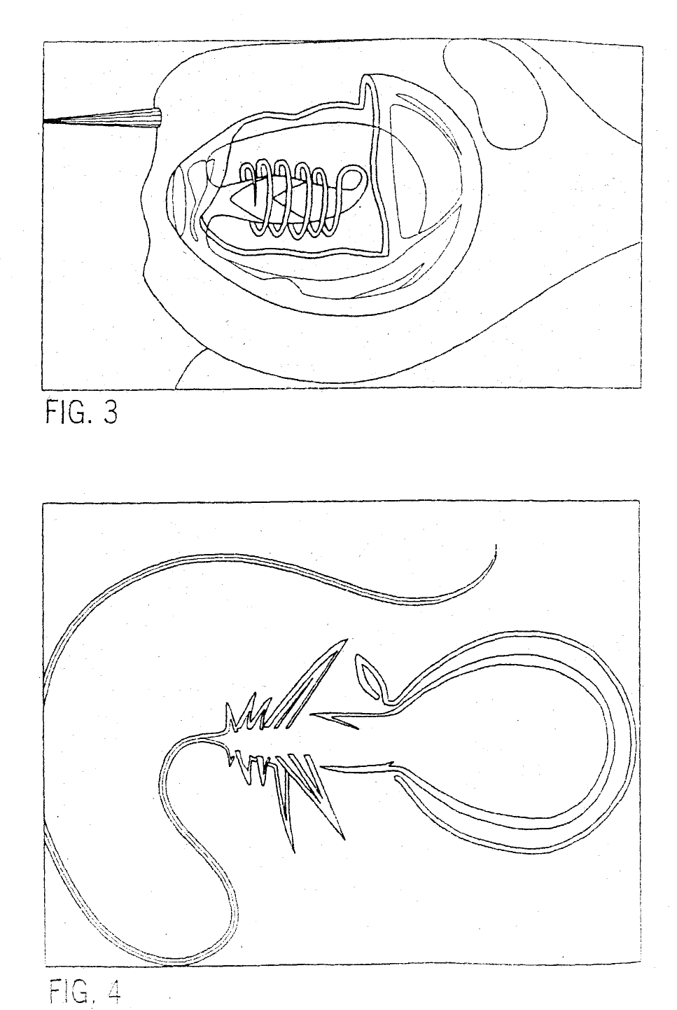

In some species, the tube can include a hole in the target end for delivery of venom to the target. Many nematocyst-bearing cnidarians also have long structures, commonly referred to as tentacles, armed with a multitude of nematocysts that can entangle around food or an attacker, causing a multitude of stings from the individual nematocysts. Furthermore, when multiple nematocysts fire the harpoon-like tubes into a target, the tubes become attached to the target and can prevent detachment of the tentacle leaving the target vulnerable to attacks from unfired nematocysts. FIGS. 2 and 3 illustrate an armed nematocyst 200 and a discharged nematocyst 210 respectively.

When nematocyst-bearing creatures sting a human, typically the human's epidermis is affected by the nematocyst. Additionally, some stings by nematocysts can penetrate multiple layers of the human epidermis. As illustrated in FIG. 4, the epidermis 100 has five distinct layers. The first layer 110 is the stratum corneum. The second layer 120 is the stratum lucidum. The third layer 130 is the stratum granulosum. The forth layer 140 is the stratum spinosum. The fifth layer 150 is the stratum basale. Additionally, the average human epidermis 100 has a typical thickness ranging from 70 to 120 microns, with an average thickness of 100 microns.

A abrasive pad 300 that is saturated with treatment solution 310 is illustrated in FIG. 5. The pad 300 includes an abrasive area, which may be present on one side 320, or may be present on second side 330. The sides 320 and 330 can differ from one another. For example, one side can be a rough, relatively more abrasive side while the other side can be relatively less abrasive. One of the sides can be smooth.

The pad 300 may be formed of a single layer of abrasive material. Alternatively, the pad 300 may be multilayered, with an absorbent material backing a porous abrasive material. The absorbent material can carry and hold a solution of a treatment substance for subsequently delivery of the solution. Thus, the treatment substance can be delivered through the abrasive material on the application of a slight pressure that can force a portion of the solution out of the absorbent material. In such an arrangement, the pad may be pre-saturated with a treatment solution, and may be stored in a sealed watertight container to prevent drying out of the pad during storage.

An alternative arrangement (not shown) can include a pad having a bladder or other receptacle either alone or in combination with the abrasive and/or absorbent material. The application of slight pressure can be used to rupture the bladder or receptacle in order to release the treatment substance into the pad. The pad could take the form of a three-layer pad, with the three layers comprising an abrasive material on one side of the pad, an absorbent material on the other side of the pad, and the central layer being formed of the bladder. A plurality of bladders can be provided, with each bladder containing a different solution that may be mixed by rupturing the bladders, either in a predetermined sequence, or all at once. The bladders can prevent deterioration of the active ingredients of the treatment solution, and can also prevent the treatment solution from evaporating.

The pad can include multiple surfaces having varying degrees of abrasiveness according to a particular use or application. A pad with one side more abrasive than the other can be beneficial so that a first, more abrasive side may be used first, followed by a second, less abrasive side. The texture of the pad may be in an amorphous, unpatterned, construction which facilitates extraction of envenomating stinging cells such as nematocysts through entanglement.

The abrasive pad may be formed from matted or entangled non-woven fibers, or may be formed from a loop fabric similar to the loop portion of hook-and-loop fasteners (Velcro) and the like. Alternatively, the abrasive pad may contain a plurality of apertures, forming a sponge-like body. The apertures or areas between fibers of the pad may have a non-uniform size and distribution, or may all be of the same size and uniformly distributed. The size of the apertures or areas between fibers of the pad are preferably of a size to enable at least part of the stinging cell to penetrate into the aperture when the pad is brought into contact with the wound, such that further movement of the pad causes the stinging cell to become entangled in the pad and hence removed from the wound with the pad. Multiple passages of the pad over the wound may entangle most or all of the stinging cells into the pad, thus removing the stinging cells from the wound. The pad may also be formed of a plastic material, or any other material of a fibrous or abrasive nature. The pad should be sufficiently abrasive so that surface layers of the skin may be removed with the pad.

The abrasive pad can be used to remove any remnants and pieces of stinging cells or tentacles still in or on the wound. The pad can be flexible and can be used as a means for removing the tentacular remnants with their undischarged nematocysts. Typically at least the stratum corneum is removed or abraded along with the tentacular remnants and undischarged nematocysts. It will be appreciated that not all stings will leave a stinger or stinging cells in the wound, particularly if caused by fire ants and bees. However, the pad may still be used to clean the wound and remove at least the stratum corneum of the skin. The treatment solution may then be easily delivered into the remaining layers of the skin.

In one embodiment, the pad may be formed into a mitt, which may be elbow length. The mitt may be used to exfoliate and debride the wound, provide a reservoir of detoxifying solution and to provide a means of delivery of the detoxifying solution. The mitt can be lined with a layer of plastic that can prevent the penetration of stinging cells and thus prevent the wearer of the mitt from being stung. The mitt may be similarly saturated with the previously prepared solution, such as a proteolytic enzyme, and aloe. The mitt can also be used to remove existing tentacles still on the patient, while protecting the wearer of the mitt from undischarged nematocysts.

The mitt may be especially useful for use with very toxic venom, such as that produced by the box jellyfish, also referred to as Chironex. The box jellyfish has nematocysts which can penetrate to a depth of 2-3 mm. Because of this depth of penetration and the deadly toxicity of the venom, extreme care must be taken in treating these victims to prevent the caregiver from being victimized by the same creature. Thus a mitt that is not penetrable by the nematocysts and covers both the hand and a portion of the arm of the caregiver can be useful. The mitt may have the highest degree of abrasion that is medically acceptable. Additionally, the mitt should be sufficiently supple to allow rounded and not readily accessible areas of the human body to be treated with at least some degree of flexion of the mitt.

In one arrangement, the pad contains a treatment solution for the treatment of animal and marine stings including 65.0% to 98.0% aloe vera and 0.1% to 20.0% of at least one proteolytic enzyme. The aloe vera provides a soothing and healing action to an inflamed and infected area of the skin after it has been stung. Aloe vera provides numerous beneficial properties such as antiseptic, anti-inflammatory, and other properties that can improve the condition of an inflamed and irritated region of tissue. Additionally, the aloe vera acts as a carrier for the proteolytic enzyme so that no additional water is needed. A carrier that is substantially without additional water can be useful when using the solution on nematocysts or other stings that leave un-discharged stinging cells at the infected site, such as the tentacles of a jellyfish that typically attach to a victim. In some circumstances, water can activate un-discharged stinging cells, causing them to fire and cause further stinging. In one arrangement, the treatment solution can also be made of 90.0% to 98.0% aloe vera, and preferably 93.0% aloe vera.

The proteolytic enzyme can neutralize the venom released by the nematocysts. In one example, the proteolytic enzyme can be papain, bromelain, peptidase, protease, trypsin, chymo-trypsin, or various combinations of these enzymes; however, other proteolytic enzymes that are suitable for such treatment can also be used. It should be noted that proteolytic enzymes may be synthetically created and may thus have names which are not necessarily associated with the original enzyme from which they were synthesized.

The proteolytic enzyme in the solutions may range in strength from 0.1% to 20% by weight. Such a percentage can be effective in treating a variety of stings; nevertheless, a greater range of a proteolytic enzyme can be used when beneficial, such as for stings by particular organisms having venom of higher toxicity. The reason for the wide range is due to the differing degrees of toxicity caused by the different creatures. For example, a lesser jellyfish such as Aueralias, known as Moon Jelly, has a reduced toxicity, and a strength of 0.1% proteolytic enzyme solution may adequately neutralize the toxin in a wound. A more toxic jellyfish such as Chironex Fleckerii, the deadly Box Jellyfish of Australia may require a 20% proteolytic enzyme solution for effectiveness.

A 0.5% to 5% of proteolytic enzyme range will generally treat most envehomations, and a range of 0.9% to 1.1% of the proteolytic enzyme is the preferred range. A 20% solution is generally the upper range of strength found to be effective without causing injury to the patient, and with an established history of dermatological application. In certain circumstances, however, it may be appropriate to use a solution having an amount of proteolytic enzyme higher than 20%. It should be noted that a very high strength solution would generally require a physician's prescription, as it could burn young or sensitive skin.

In some embodiments, the aloe vera and proteolytic enzyme solution can be mixed with other substances. The treatment solution can include 1.0% to 10.0% of at least one humectant, such as glycerin, that promotes the retention of moisture. Alternatively, around 4% of the treatment solution can be dipropylene glycol which can be used as a humectant, a softening agent, an ointment base, a preservative, and an emollient. Dipropylene glycol can improve the absorption of the protective ingredients by the skin. Additionally, around 7% to 10% of the solution can include sodium xylenesulfonate as a hydrotropic solvent.

The solution can also include at least 0.1% to 5.0% of at least one viscosity agent, such as sodium chloride or xanthan gum. In one arrangement, the solution can include 0.25% xanthan gum and 1.0% sodium chloride to obtain a preferred viscosity. Sodium chloride also provides the added benefit of further preventing activation of non-discharged stinging cells.

The treatment solution can also include 0.05% to 1.1% of at least one pH stabilizer, such as aminomethylpropanol. Preferably, the solution can include 0.1% of aminomethylpropanol to provide a pH in the range of about 6.5 to 8.0. The solution can also include 0.05% to 1.1% of at least one anti-bacterial agent such as diazolindinyl urea and Triclosan (registered trademark). The treatment solution can also include 0.05% to 0.25% of at least one fungal agent, such as methlyparaben at 0.2% and propylparaben at 0.1%. Alcohol such as methanol and a surfactant, such as sodium lauryl ether sulfate, can be included. An antitoxin and an antiseptic can also be included.

Three exemplary formulations of the treatment solution are listed below; however, it should be noted that the invention is not limited to the formulations presented and that any suitable formulation can be used.

### EXAMPLE 1

### Solution for Treating Flying Insect Stings

| INGREDIENTS: | % |
|---|---|
| ALOE VERA GEL | 70.96869 |
| DIPROPYLENE GLYCOL | 4.00000 |
| TRICLOSAN | 1.50000 |
| SODIUM XYLENESULFONATE | 8.40000 |
| SODIUM LAURYL ETHER SULFATE | 13.82000 |
| PAPAIN | 1.00000 |
| FRAGRANCE | 0.30000 |
| FD&C BLUE #1 | 0.005655 |
| FD&C YELLOW #5 | 0.005655 |

### EXAMPLE 2

### Solution for Treating Fire Ant Stings

| INGREDIENTS: | % |
|---|---|
| ALOE VERA GEL | 70.97435 |
| DIPROPYLENE GLYCOL | 4.00000 |
| TRICLOSAN | 1.50000 |
| SODIUM XYLENESULFONATE | 8.40000 |
| SODIUM LAURYL ETHER SULFATE | 13.82000 |
| PAPAIN | 1.00000 |
| FRAGRANCE | 0.30000 |
| FD&C BLUE #6 | 0.005655 |

### EXAMPLE 3

### Solution for Treating Marine Specie Stings

| INGREDIENTS: | % |
|---|---|
| ALOE VERA GEL | 93.43869 |
| GLYCERIN | 3.00000 |
| XANTHAN GUM | 0.25000 |
| SODIUM CHLORIDE | 1.00000 |
| AMINOMETHYLPROPANOL | 0.10000 |
| PAPAIN | 1.00000 |
| FRAGRANCE | 0.20000 |
| PROPYLENE GLYCOL | 0.56000 |
| DIAZOLIDINYL UREA | 0.14000 |
| METHYLPARABEN | 0.20000 |
| PROPYLPARABEN | 0.10000 |
| FD&C BLUE #1 | 0.005655 |
| FD&C YELLOW #5 | 0.005655 |

Testing of a pad saturated with a solution according to the present invention, showed the successful use of a pad and method according to the invention on the marine and land species identified in Table 1 below.

TABLE 1

| CLASSIFICATION | COMMON NAME | DISTRIBUTION |
|---|---|---|
| PORIFERA | | |
| Family Desmacidonae | Red Moss Sponge | U.S. Cape Cod to S. Carolina, |
| Family Tendaniidae | Fire Sponge | West Indies |
| COLENTERATA | | |
| Hydrozoans | | |
| Family Milliporidae | Stinging and/or Fire Coral | Tropical Pacific and Indian |
| | | Oceans, Red Sea, Caribbean |
| | | Tropical Pacific and Indian |
| Family Plumulariidae | Stinging Seaweed | Oceans, West Indies |
| | | Atlantic Ocean, |
| Family Physaliidae | Atlantic Portuguese | Mediterranean Sea |
| | Man-O-War | Tropical Indian and Pacific |
| | Indo-Pacific | Oceans |
| | Man-O-War | |
| SCYPHOZOA | | |
| Family Chirodropidae | Deadly Sea Wasp; Box | Australian Pacific Coast |
| | Jellyfish | |
| Family Carybdeidae | Sea Wasp | Tropical Pacific, Atlantic and |
| | | Indian Oceans. |
| Family Cyaneidae | Sea Blubber; Lion's Mane | Northern Atlantic & Pacific, |
| | | Baltic Sea, Tropical and |
| | | Temperate Pacific Ocean |
| ANTHOZOA | Sea Anemones & Coral | World-Wide |
| Family Acroporidae | Elkhorn Coral | Caribbean Sea |
| Family Actiniidae | Sea Anemone | Eastern Atlantic, |
| | | Mediterranean, Black Sea |
| | | and Sea of Asia |
| Family Actinodendronidae | Hell's Fire Sea Anemone | Atlantic and Pacific Oceans |
| Family Aliciidae | Sea Anemone | Red Sea |
| Family Hormathiidae | Sea Anemone | Eastern Atlantic, |
| | | Mediterranean Sea, Coastal |
| | | Africa |
| ANNELIDA | Segmented Worms | World-Wide |
| Family Amphinomidae | Fire Worm | Gulf of Mexico, Tropical |
| | | Pacific and Atlantic Oceans |
| ENCHINODERMATA | Starfish, Sea Cucumbers, | World-Wide |
| | Sea Urchins | |
| Class Holothuroidae | Sea Cucumber | All temperate bodies of water |
| | | Pacific and Indian Oceans |
| Family Toxopneustidae | Sea Urchin | |
| ECTOPROCTA | | |
| Family Alcyonidioidae | Bryozoans | World-Wide |
| ARTHROPODIA | Spiders and Insects | World-wide |
| Myrmicinae | Ants | World-Wide |
| Solenopsis | Fire Ants and other ants | World-Wide |
| Pogonomyrmex | | |
| Paraponera | | |
| Diptera and Acarina | No-see-ums, punkies, gnats, | World-Wide |
| Nematocera | moose flies, midges, biting | |
| Ceratopogonidae | midges, blood sucking gnats, | |
| Culicoides | mites, chigoe, jigger, chigger, | |
| Acari | fleas, blow flies, blue bottle, | |
| Culex | biting housefly, stable fly, | |
| Trichoceridae | buffalo gnats, black flies | |
| Mycetophilidae | | |
| Chaoborinae | | |
| Calliphoridae | | |
| Simuliidae | | |

It should be understood that the examples and embodiments described herein are for illustrative purposes only and that various modifications or changes in light thereof will be obvious to persons skilled in the art, and that such modifications or changes are to be included within the spirit and purview of this application. Moreover, the invention can take other specific forms without departing from the spirit or essential attributes thereof.

What is claimed is:

1. An abrasive pad for delivering a skin treatment for the treatment of animal and marine stings, comprising:
a material having an abrasive surface, wherein said surface is sufficiently abrasive to mechanically abrade at least the stratum corneum from a patient's skin; and
a treatment solution applied to said material for contact with the patient's skin, wherein said treatment solution comprises a solution of 65.0% to 98.0% aloe vera and 0.1% to 20.0% of at least one proteolytic enzyme and said treatment solution contains substantially no added water.

2. The abrasive pad according to claim 1, wherein said pad is multilayered.

3. The abrasive pad according to claim 1, wherein said pad comprises an absorbent material carrying said treatment solution, and a porous abrasive material.

4. The abrasive pad according to claim 1, wherein said pad comprises a burstable receptacle containing said treatment solution.

5. The abrasive pad according to claim 1, wherein said pad comprises three layers including two outer layers and a central layer, at least one of the outer layers being of an abrasive material, and the burstable receptacle forming the central layer.

6. The abrasive pad according to claim 1, wherein a plurality of burstable receptacles are provided, at least one of said burstable receptacles containing a different treatment solution from at least one other of said burstable receptacles.

7. The abrasive pad according to claim 1, wherein said abrasive surface is formed of matted non-woven fibers.

8. The abrasive pad according to claim 1, wherein said abrasive surface is formed of a loop fabric.

9. The abrasive pad according to claim 1, wherein said abrasive surface is sponge-like.

10. The abrasive pad according to claim 1, wherein said pad includes a mitt adapted to be worn over a hand of a user.

11. The abrasive pad according to claim 1, wherein 0.1% to 5.0% of said solution comprises said at least one proteolytic enzyme.

12. The abrasive pad according to claim 11, wherein 0.9% to 1.1% of said solution comprises said at least one proteolytic enzyme.

13. The abrasive pad according to claim 1, wherein said at least one proteolytic enzyme is papain.

14. The abrasive pad according to claim 1, wherein 90.0% to 98% of said solution comprises aloe vera.

15. The abrasive pad according to claim 1, wherein 1.0% to 10.0% of said solution comprises at least one humectant.

16. The abrasive pad according to claim 15, wherein said at least one humectant is glycerin.

17. The abrasive pad according to claim 1, wherein 0.1% to 5.0% of said solution comprises at least one viscosity agent.

18. The abrasive pad according to claim 17, wherein said at least one viscosity agent is at least one of sodium chloride and xanthan gum.

19. The abrasive pad according to claim 1, wherein 0.05% to 1.0% of said solution comprises at least one pH stabilizer.

20. The abrasive pad according to claim 19, wherein said pH stabilizer is aminomethylpropanol.

21. The abrasive pad according to claim 1, wherein 0.05% to 1.1 % of said solution comprises at least one anti-bacterial agent.

22. The abrasive pad according to claim 21, wherein said at least one anti-bacterial agent is at least one of diazolidinyl urea and triclosan.

23. The abrasive pad according to claim 1, wherein 0.05% to 0.25% of said solution comprises at least one fungal agent.

24. The abrasive pad according to claim 23, wherein said at least one fungal agent is at least one of methylparaben and propylparaben.

25. The abrasive pad according to claim 1, wherein said solution comprises at least one surfactant.

26. The abrasive pad according to claim 1, wherein said solution comprises at least one antitoxin.

27. The abrasive pad according to claim 1, wherein said solution comprises at least one antiseptic.

28. The abrasive pad according to claim 1, wherein said solution comprises at least one alcohol.

29. The abrasive pad according to claim 1, wherein the sting has been caused by an arthropod.

30. The abrasive pad according to claim 29, wherein the arthropod is Solenopsis Invicta.

31. The abrasive pad according to claim 1, wherein the sting was caused by a cnidarian.

32. The abrasive pad according to claim 31, wherein the cnidarian is a larval of the thimble jellyfish.

33. A solution for treating animal and marine stings, comprising:
65.0% to 98.0% aloe vera; and
0.1 % to 20.0% of at least one proteolytic enzyme;
wherein said solution contains substantially no added water.

34. The solution according to claim 33, wherein 0.1% to 5.0% of said solution comprises said at least one proteolytic enzyme.

35. The solution according to claim 34, wherein 0.9% to 1.1% of said solution comprises said at least one proteolytic enzyme.

36. The solution according to claim 33, wherein said at least one proteolytic enzyme is papain.

37. The solution according to claim 33, wherein 90.0% to 98% of said solution comprises aloe vera.

38. The solution according to claim 33, wherein 1.0% to 10.0% of said solution comprises at least one humectant.

39. The solution according to claim 38, wherein said at least one humectant is glycerin.

40. The solution according to claim 33, wherein 0.1% to 5.0% of said solution comprises at least one viscosity agent.

41. The solution according to claim 40, wherein said at least one viscosity agent is at least one of sodium chloride and xanthan gum.

42. The solution according to claim 33, wherein 0.05% to 1.0% of said solution comprises at least one pH stabilizer.

43. The solution according to claim 42, wherein said at least one pH stabilizer is aminomethylpropanol.

44. The solution according to claim 33, wherein 0.05% to 1.1% of said solution comprises at least one anti-bacterial agent.

45. The solution according to claim 44, wherein said at least one anti-bacterial agent is at least one of diazolidinyl urea and triclosan.

46. The solution according to claim 33, wherein 0.05% to 0.25% of said solution comprises at least one fungal agent.

47. The solution according to claim 46, wherein said at least one fungal agent is at least one of methylparaben and propylparaben.

48. The solution according to claim 33, wherein said solution comprises at least one surfactant.

49. The solution according to claim 33, wherein said solution comprises at least one antitoxin.

50. The solution according to claim 33, wherein said solution comprises at least one antiseptic.

51. The solution according to claim 33, wherein said solution comprises at least one alcohol.
